(19) United States
(12) Patent Application Publication
(10) Pub. No.: US 2005/0038443 A1
(43) Pub. Date: Feb. 17, 2005

(54) Surgical tools for joint replacement

(76) Inventors: Anthony K. Hedley, Phoenix, AZ (US); Michael Howard, Prescott, AZ (US); Henry H. Fletcher, Cameron Park, CA (US)

Correspondence Address: ARTHUR J. BEHIEL, 6601 KOLL CENTER PARKWAY, SUITE 245, PLEASANTON, CA, 94566, US

(21) Appl. No.: 10/734,330

(22) Filed: Dec. 11, 2003

Related U.S. Application Data
(60) Provisional application No. 60/432,730, filed on Dec. 12, 2002.
(60) Provisional application No. 60/432,729, filed on Dec. 12, 2002.
(60) Provisional application No. 60/474,366, filed on May 30, 2003.

(51) Int. Cl. A61F 5/04; A61B 17/56; A61F 2/32; A61B 17/58; A61B 17/60

(52) U.S. Cl. 606/91; 606/86; 606/53

(57) ABSTRACT

Described are surgical tools, including tool drivers and implantation instruments, that provide improved visual and positional access to human acetabulum. Some embodiments include a conduit with multiple bends to circumvent soft tissue surrounding the acetabulum. The conduits may employ a number of interlocking, rotational links to transfer torque from a drive end of the tool to a bit end. In one embodiment the bit end supports an attachment actuator that securely engages a conventional acetabular cup for insertion and placement. The attachment actuator can release the cup without moving the body of the tool, which prevents accidental dislodging of a properly placed acetabular cup.

### BACKGROUND

FIG. 1 (prior art) depicts an acetabular reamer cup 100, a type of surgical bit used to cut precisely sized hemispherical cavities in the human acetabulum, a cavity at the base of the hipbone into which fits the ball-shaped head of the femur. Acetabular reamer cups are generally mounted on a tool driver via a pair of cross members 105. The tool driver is in turn mounted in the chuck or collet of a low-speed, high torque portable drill or flexible powered shaft. An embodiment of reamer cup 100 is detailed in U.S. Pat. No. 6,428,543, which is incorporated herein by reference.

FIG. 2 (prior art) is a cross section of a joint-replacement cup 200, in this example an acetabular cup, for implanting into a hemispherical cavity formed using reamer cup 100. Acetabular cup 200 becomes part of an artificial hip joint. A threaded hole 205 firmly secures the concave inner surface 210 of cup 200 against an implantation instrument (not shown) used to insert and position cup 200 into the associated cavity.

Soft tissue surrounds the acetabulum, and interferes with tool drivers and implantation instruments. This problem is exacerbated in larger patients, who disproportionately require hip-replacement surgery. There is therefore a need for tool drivers and implantation instruments that provide improved access to the acetabulum.

For detailed discussions of hip replacement, including tool drivers and implantation instruments, see U.S. Pat. Nos. 5,320,625; 6,428,543; and 5,817,096; which are incorporated herein by reference.

### SUMMARY

The present invention is directed to surgical tools, including tool drivers and implantation instruments, that provide improved visual and positional access during joint-replacement surgery. Tool drivers and implantation instruments in some embodiments include multiple bends to circumvent soft tissue surrounding the acetabulum. The tool and drive ends may extend along parallel axes so tool operators enjoy a correct sense of reamer or cup placement.

Tool drivers with one or more bends provide improved access, but the bends complicate the task of transmitting high torque from the drive end to the tool end. Some embodiments address this problem using a drive mechanism made up of a number of interlocking, rotational links.

A hip-replacement tool in accordance with another embodiment supports an attachment actuator that securely engages a conventional acetabular cup for insertion and placement. The attachment actuator supports an attach state and a release state. In the attach state, threaded jaws in the attachment actuator expand into a hole in the acetabular cup. In the release state, the threaded jaws contract to disengage the cup without rotating with respect to the cup. Users can control the states of the attachment actuator without moving the body of the tool, so tool operators can detach the tool from the implanted cup without accidentally dislodging or misaligning the cup.

This summary does not limit the invention, which is instead defined by the claims.

### BRIEF DESCRIPTION OF THE FIGURES

FIG. 1 (prior art) depicts an acetabular reamer cup 100, a type of surgical bit used to cut precisely sized hemispherical cavities in the human acetabulum.

FIG. 2 (prior art) is a cross section of an acetabular cup 200 for implanting into the hemispherical cavities formed using reamer cup 100.

FIG. 3 is a side view of a hip-replacement tool 300 in accordance with one embodiment.

FIG. 4 depicts an embodiment of tool 300 of FIG. 3 in cross section, with like-numbered elements being the same as those of FIG. 3.

FIG. 5 depicts a portion of conduit 305 in cross section, detailing a number of interlocking rotational links 405.

FIG. 6A depicts link 405 from a perspective facing male end 510.

FIG. 6B depicts a link 405 from a perspective facing female end 515.

FIG. 7 depicts a link 700 in accordance with another embodiment.

FIG. 8 depicts a link 800 in accordance with another embodiment.

FIG. 9 depicts a hip-replacement tool 900 in accordance with an embodiment used for implanting and positioning an acetabular cup, such as cup 200 of FIG. 2.

FIG. 10 depicts bit end 910 of tool 900 in more detail for ease of illustration.

FIG. 11 depicts end 910 of tool 900 with cup attachment 920 removed from conduit 905 to better illustrate actuator 1000.

FIG. 12 is a cross-section of cup attachment 920 in accordance with one embodiment.

FIG. 13 depicts an embodiment of tool 900 of FIG. 9 in cross section.

### DETAILED DESCRIPTION

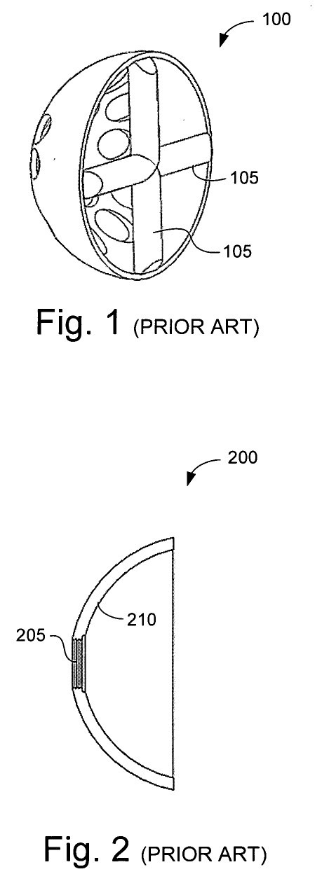

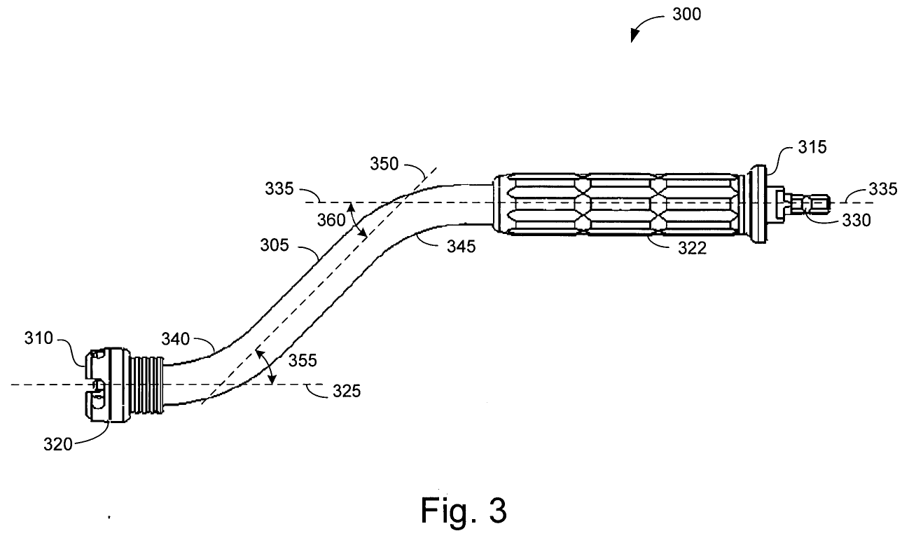

FIG. 3 is a side view of a surgical tool 300 in accordance with one embodiment. Tool 300, a hip-replacement tool in this example, includes a conduit 305 extending between a bit end 310 and a drive end 315. Bit end 310 supports a head 320 that rotates with respect to conduit 305 on a first axis 325. Drive end 315 includes a handle 322. A shaft end 330 adapted to mate with a drill collet extends from drive end 315, and rotates on a second axis 335. In one embodiment, a flexible shaft extends through conduit 305 from shaft end 330 to head 320, so rotating shaft end 330 similarly rotates head 320. Head 320 mates with an acetabular reamer cup similar to cup 100 of FIG. 1, and is, in this embodiment, of a type described in U.S. Pat. Nos. 6,540,739 and 6,506,000, both of which are incorporated herein by reference.

Conduit 305 includes a pair of bends 340 and 345, so a portion of conduit 305 extends along a third axis 350 at an angle 355 with respect to first rotational axis 325 and an angle 360 with respect to second rotational axis 335. Angles 355 and 360 are equal in the depicted embodiment, though this need not be the case. The double bend of tool 300 avoids soft tissue for improved visibility and positional accuracy, but still provides a straight-line approach to tool placement. In embodiments in which rotational axes 325 and 335 are parallel, the operator enjoys a correct sense of the position of bit end 310 even when blood and tissue obstruct direct viewing.

The inclusion of bends 340 and 345 facilitates ease of access, but renders difficult the task of transmitting high torque through conduit 305. Some embodiments employ a flexible shaft to convey torque from shaft end 330 to head 320, but such embodiments sometimes suffer gripping and vibration when actuating an acetabular reamer cup against hard or uneven bone surfaces.

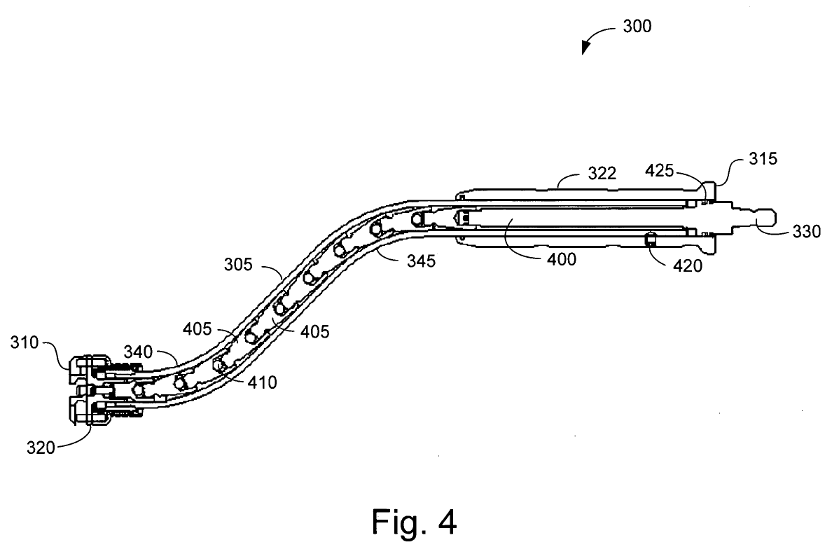

FIG. 4 depicts an embodiment of tool 300 of FIG. 3 in cross section, with like-numbered elements being the same as those of FIG. 3. (In general, this document uses a numbering convention in which the leading digit or digits identifies the figure in which the element was introduced.) Rotating head 320 connects to shaft end 330 via a drive shaft 400 and a number of interlocking rotational links 405. Bushings 410 are disposed between adjacent links 405. The embodiment of FIG. 4 has been found to transfer torque more evenly than flexible shafts.

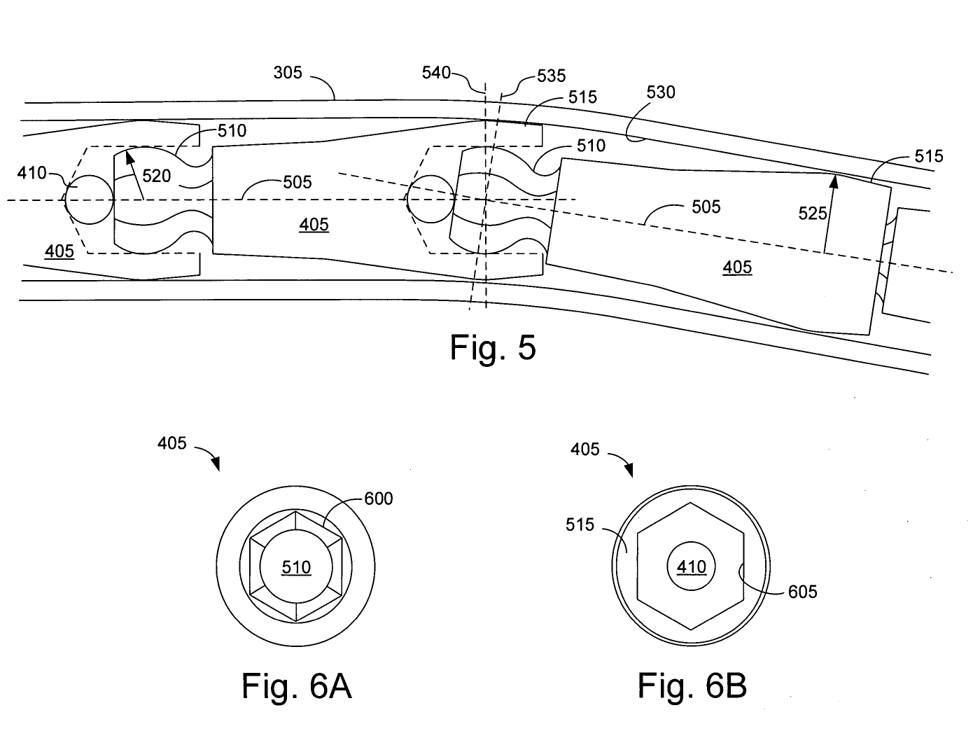

FIG. 5 depicts a portion of conduit 305 in cross section, detailing a number of interlocking rotational links 405. Each link 405 is symmetrical about a respective link access 505, and includes a male end 510 and a female end 515. Male end 510 has a radius of curvature 520 that allows each link 405 to pivot in a plane parallel to link axes 505 within female end 515 in an adjacent link 405. The exterior surface of each link 405 includes a radius of curvature 525 that allows the female end of each link 405 to pivot in a plane parallel to link axes 505 and freely against the interior wall 530 of conduit 305.

Referring to the interconnection of the two full links of FIG. 5, a dashed line 535 extends through the pivotal axis of male end 510 and a dashed line 540 extends through the pivotal axis of female end 515. The intervening bushing 410 maintains the intersection of the two pivotal axes over a range of angles. In other words, the pivotal axes of the male and female ends remain substantially coaxial when the rotational axes 505 of adjacent links 405 are misaligned. This link arrangement prevents links 405 from binding against one another and against interior wall 530 when transmitting torque around bends in conduit 305.

FIG. 6A depicts link 405 from a perspective facing male end 510. In this embodiment, link 405 includes six exterior facets 600, though other shapes might be used. FIG. 6B depicts a link 405 from a perspective facing female end 515. Female end 515 includes six interior facets 605 that mate with the exterior facets 600 of an adjacent link 405.

In one embodiment, conduit 305 is a 416 stainless-steel pipe with an inside diameter of about 0.410 inches and an outside diameter of about 0.625 inches. Each of bends 340 and 345 is about forty five degrees, with a bend radius of about 2.18 inches. In one embodiment, conduit 305 is formed by drilling out a 416 stainless-steel rod, forming bends 340 and 345, forcing appropriately sized spheres through the resulting channel to restore the inside diameter within curves 340 and 345 using a hydraulic press, and hardening the resulting conduit. The hardened 416 stainless steel advantageously provides an excellent bearing surface for links 410. Links 410 are, in one embodiment, machined from 440-C stainless steel.

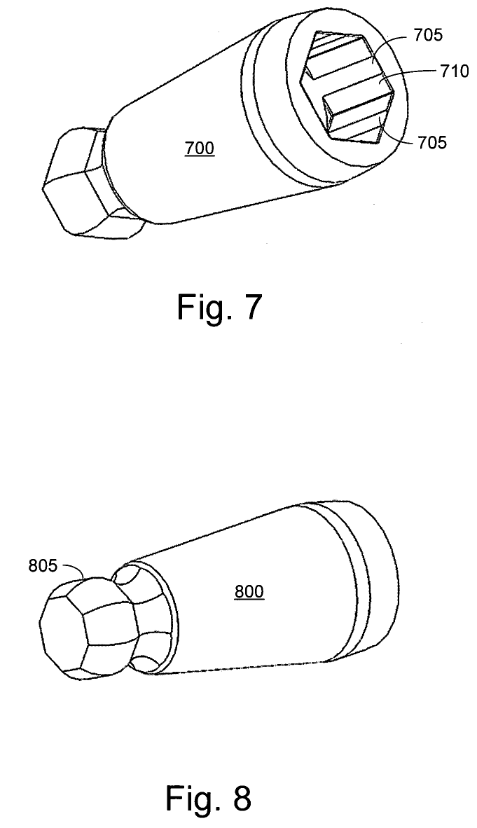

FIG. 7 depicts a link 700 in accordance with another embodiment. Link 700 is similar to links 410 of FIG. 4, but includes a lubrication channel 705 in one or more of interior facets 710. In one embodiment, lubrication channels 705 are formed by first pre-drilling the female end of line 700 to include round hole slightly larger in diameter then the short dimension of the hexagonal hole to be formed in the female end. The corners of the hexagon are then formed either by stamping the hole with a hexagonal dye and removing the resulting chips or using a conventional wobbling broach technique.

FIG. 8 depicts a link 800 in accordance with another embodiment. Link 800 is similar to link 700 of FIG. 7, but includes 8 exterior facets 805 and eight interior facets (not shown).

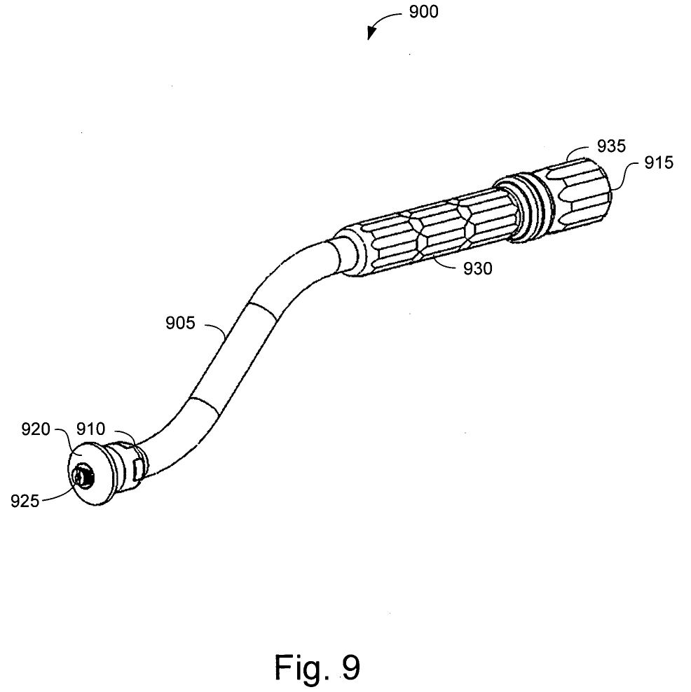

FIG. 9 depicts a surgical tool 900 in accordance with an embodiment used for implanting and positioning a cup, such as acetabular cup 200 of FIG. 2. Tool 900 includes a conduit 905 extending between a bit end 910 and a handle end 915. Bit end 910 supports a cup attachment 920 through which protrudes a pair of jaws 925 adapted to extend into and engage with hole 205 of cup 200 (FIG. 2). As detailed below, jaws 925 are parts of an attachment actuator that supports an attach state and a release state: the attach state secures tool 900 to acetabular cup 200 and the release state releases cup 200. A user controls the states of the attachment actuator by grasping a knurled handle 930 and rotating a knob 935 on drive end 915. Tool 900 can release cup 200 while holding conduit 905 and handle 930 still, which prevents accidental dislodging of a properly placed cup 200. As in tool 300 of FIG. 3, the inclusion of two bends in tool 900 provides improved visual and surgical access, particularly for relatively large patients.

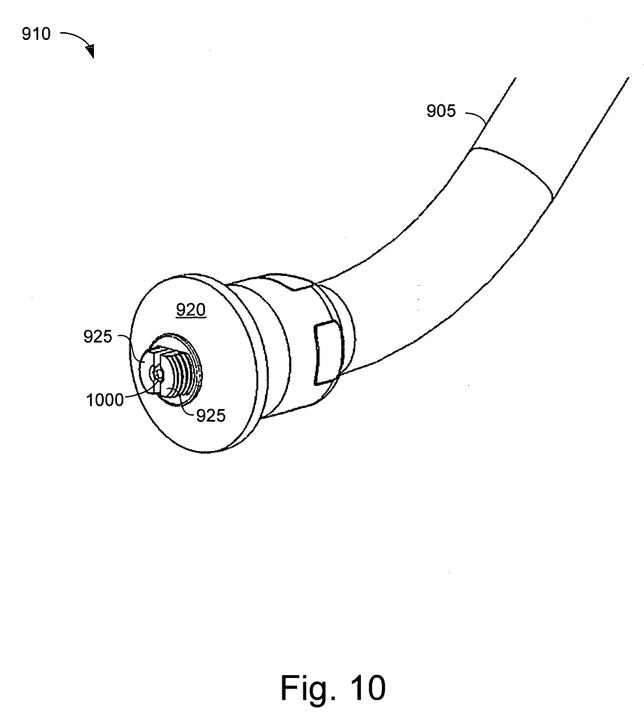

FIG. 10 depicts bit end 910 of tool 900 in more detail for ease of illustration. An actuator 1000 extends between jaws 925. Rotating knob 935 clockwise with respect to handle 930 extends actuator 1000 outward, spreading jaws 925; conversely, rotating knob 935 counter-clockwise withdraws actuator 1000, allowing jaws 925 to close.

Jaws 925 each include thread portions 1005 sized to engage the female threads of hole 205 in cup 200. Cup 200 can thus be mounted on cup attachment 920 either rotationally (taking advantage of thread portions 1005) or by extending jaws 925 through hole 205 in the release state and turning knob 935 to spread jaws 925 to engage threaded portions 1005. Tool 900 can then be used to position, implant, and adjust cup 200.

Once cup 200 is properly placed, tool 900 can easily release cup 200 without disturbing the position of cup 200. Rotating knob 935 counter-clockwise withdraws actuator 1000, allowing jaws 935 to close and release cup 200. The ability of tool 900 to maintain a secure hold on cup 200 is important, as positioning and implanting cup 200 can require considerable force, possibly even hammer blows on knob 935. The ability of tool 900 to gently release cup 200 is also important, as cup 200, once properly positioned, should not be disturbed. Conventional tools that rely upon a rotational connection to threads 205 sometimes cross thread, rendering removal difficult and posing a danger of cup displacement.

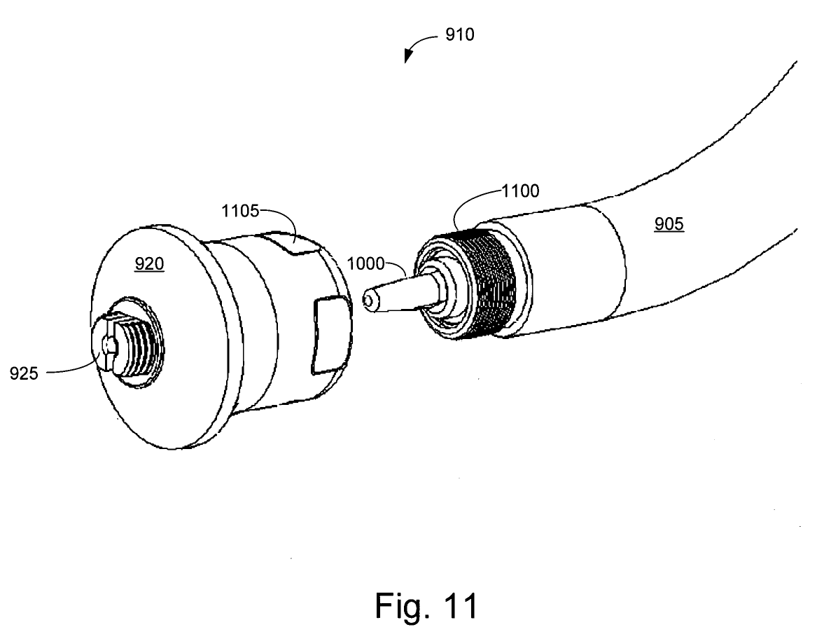

FIG. 11 depicts end 910 of tool 900 with cup attachment 920 removed from conduit 905 to better illustrate actuator 1000. Cup attachment 920 mates with threads 1100 on conduit 905, and includes facets 1105 for accepting a suitable wrench.

Actuator 1000 moves in and out of conduit 905 with rotation of knob 935. Actuator 1000 mates with interior threads (not shown) within conduit 905. In one embodiment, the threads on actuator 1000 and the corresponding threads 905 are so-called double threads. Instead of a single helical land, as in most conventional threads, double threads have two interlaced helical lands, rather like the stripes of a barber pole. Double threads advance a mating threaded component twice as far in one turn as a single thread.

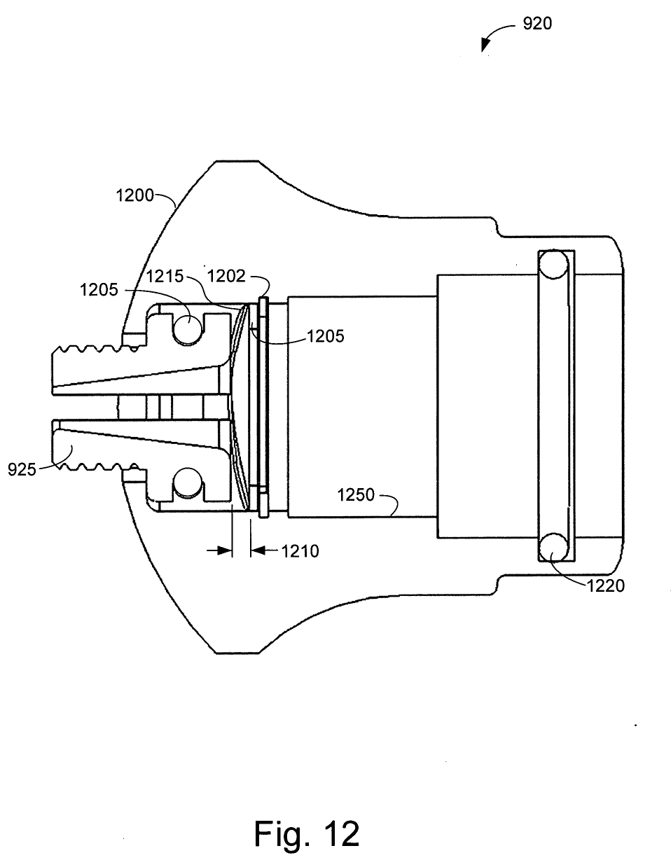

FIG. 12 is a cross-section of cup attachment 920 in accordance with one embodiment. Jaws 925 extend out through the face 1200 of cup attachment 920 and are held in place by a retaining ring 1202, a washer 1205, and a spring 1215 (spring 1215 is a Belleville washer in one embodiment). An O-ring 1220 urges jaws 925 against actuator 1000 (FIG. 10) so that jaws 925 close as actuator 1000 is withdrawn. Spring 1215 forces jaws 925 out through face 1200 of cup attachment 920. A gap 1210 between jaws 925 and washer 1205 prevents jaws 925 from taking the force of hammer blows by allowing jaws 925 to recede into cup attachment 920 until face 1200 engages the interior surface of cup 200. Face 1200, and not the more fragile jaws 925 and associated drive mechanism, thus absorbs the impact. A second O ring 1220 prevents blood and debris from entering cup attachment 920 between attachment 920 and conduit 905. Though not shown here, attachment 920 includes female threads on an inside surface 1250 that mate with threads 1100 on the outside of conduit 905 (FIG. 11).

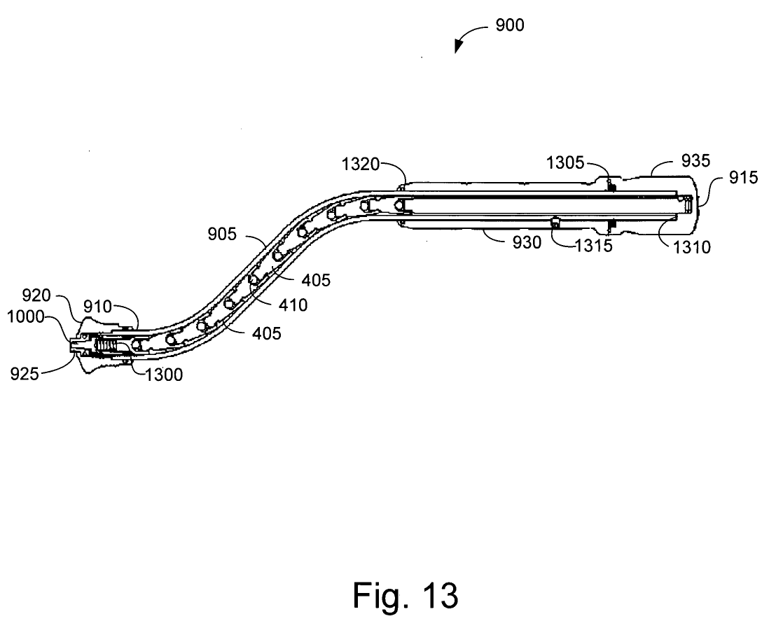

FIG. 13 depicts an embodiment of tool 900 of FIG. 9 in cross section. Various drive mechanisms can be used to force jaws 925 apart or allow jaws 925 to close. In this embodiment, however, a number of links 405 and bushings 410 of the type described above in connection with FIG. 4 transfer rotational motion of knob 935 to a threaded portion 1300 of actuator 1000. An O-ring 1305 seals knob 935 against handle 930 while allowing for relative rotation. Knob 935 includes a shoulder 1310 that rests against conduit 905. The force of blows applied to knob 935 is thus transmitted to cup attachment 920 via conduit 905, and not via the more sensitive drive mechanism. A set screw 1315 secures handle 930 to conduit 905, and an O-ring 1320 precludes blood and debris from collecting between handle 930 and conduit 905.

While the present invention has been described in connection with specific embodiments, variations of these embodiments will be obvious to those of ordinary skill in the art. For example:
a. Hip-replacement tool 900 of FIG. 9 need not have split threads, as shown, but might also include a more traditional rotating thread actuated using the disclosed link system or some other flexible means for providing torque through the channel;
b. Conduits in accordance with some embodiments are flexible to allow the bends to be adjusted over a range of angles. A series of rotational links might be installed, for example, within flexible conduits of the type available from e.g. Lockwood Products, Inc., under the trademark LOC-LINE.
c. The medical tools described above in the context of hip replacement can be used to advantage in other surgical procedures.
d. Veterinary joint replacement surgery will benefit from the tools described herein.
e. The link systems described herein have broad application outside the medical field.
f. Some embodiments can be modified to include a motor to provide the driving force.
   Therefore, the spirit and scope of the appended claims should not be limited to the foregoing description.

1. A surgical tool for manipulating a joint-replacement cup, the tool comprising:
a. a conduit having a head end and a drive end, wherein the head end is adapted to removably attach to the cup; and
b. a drive mechanism extending between the head end and the drive end, the drive mechanism rotating on a first axis at the head end and on a second axis on the drive end;
c. wherein at least a portion of the drive mechanism rotates on a third axis at a first angle with respect to the first axis and a second angle with respect to the second axis.

2. The surgical tool of claim 1, wherein the first and second angles are substantially equal.

3. The surgical tool of claim 2, wherein the first and second axes are substantially parallel.

4. The surgical tool of claim 1, wherein the cup comprises an acetabular reamer.

5. The surgical tool of claim 1, further comprising a plurality of interlocking links extending through the conduit.

6. The surgical tool of claim 1, wherein each of the links includes a male end and a female end.

7. The surgical tool of claim 6, wherein the male end includes a plurality of exterior facets and the female end includes a plurality of interior facets.

8. The surgical tool of claim 7, wherein the exterior facets define a hexagon.

9. The surgical tool of claim 7, wherein each link rotates along a link axis, and wherein the male end has a radius of curvature in a plane parallel to the rotational axis.

10. The surgical tool of claim 9, wherein the female end has a second radius of curvature in the plane.

11. The surgical tool of claim 6, further comprising a bushing disposed within the female end of a first of the links and the male end of a second of the links.

12. The surgical tool of claim 11, wherein the bearing is spherical.

13. The surgical tool of claim 1, wherein the acetabular cup is comprises a reamer surface.

14. The surgical tool of claim 1, wherein the head comprises a cup support receiving an acetabular cup.

15. A surgical tool for positioning a joint-replacement cup, the joint-replacement cup including a threaded hole, the surgical tool comprising:
a. a conduit having a head end and a drive end;
b. a drive mechanism rotatably attached to the drive end of the conduit, the drive mechanism rotating on a first axis; and
c. a head connected to the head end of the conduit, the head including:
i. a cup attachment supporting the cup; and
ii. a threaded attachment actuator having an attach state and a release state, the attach state securing the cup attachment to the cup and the release state releasing the cup;
iii. wherein the actuator support transitions between the attach and release states without rotating with respect to the conduit.

16. The surgical tool of claim 15, wherein the attachment actuator includes first and second jaws extending into the hole.

17. The surgical tool of claim 16, wherein the attachment actuator further includes a wedge extending between the first and second jaws, and wherein the attach state corresponds to a first wedge position and the release state corresponds to a second wedge position.

18. The surgical tool of claim 17, wherein the hole comprises female threads, and wherein the first and second jaws include partial threads.

19. The surgical tool of claim 18, wherein the partial threads engage the female threads in the first wedge position and disengage the female threads in the second wedge position.

20. The surgical tool of claim 15, wherein the conduit includes at least one bend between the head end and the drive end.

21. The surgical tool of claim 15, further comprising a plurality of interlocking links extending through the conduit.

22. The surgical tool of claim 15, wherein each of the links includes a male end and a female end.

23. The surgical tool of claim 22, wherein the male end includes a plurality of exterior facets and the female end includes a plurality of interior facets.

24. The surgical tool of claim 23, wherein the exterior facets define a hexagon.

25. The surgical tool of claim 23, wherein each link rotates along a link axis, and wherein the male end has a radius of curvature in a plane parallel to the rotational axis.

26. The surgical tool of claim 25, wherein the female end of each link has a second radius of curvature in the plane.

27. The surgical tool of claim 26, further comprising a bearing disposed within the female end of a first of the links and the male end of a second of the links.

28. (currently canceled).

29. (currently canceled).

30. (currently canceled).

31. (currently canceled).

32. (currently canceled).

33. (currently canceled).

34. (currently canceled).

35. (currently canceled).

36. (currently canceled).

37. (currently canceled).

38. (currently canceled).

39. (currently canceled).

40. (currently canceled).

41. (currently canceled).
